(19) United States
(12) Patent Application Publication
(10) Pub. No.: US 2022/0323670 A1
(43) Pub. Date: Oct. 13, 2022

(54) SYRINGE PREFILLED WITH PHARMACEUTICAL FLUID

(71) Applicant: Genixus, Corp., Kannapolis, NC (US)

(72) Inventor: Kendall Thomas Foster, Mooresville, NC (US)

(73) Assignee: Genixus, Corp., Kannapolis, NC (US)

(21) Appl. No.: 17/715,820

(22) Filed: Apr. 7, 2022

Related U.S. Application Data
(60) Provisional application No. 63/172,430, filed on Apr. 8, 2021.

(51) Int. Cl. A61M 5/00; A61M 5/50; A61K 31/05; B65B 55/00; B65B 31/02; B65D 77/04; B65D 81/20; B65D 81/26

(52) U.S. Cl. CPC A61M 5/002; A61M 5/5086; A61K 31/05; B65B 55/00; B65B 31/02; B65D 77/0406; B65D 81/2084; B65D 81/266; A61M 2205/6054; B65B 2220/20

(57) ABSTRACT

A prepackaged injectable pharmaceutical composition includes a sealed outer package made of a material having a low oxygen transmission rate and an inner package disposed within the sealed outer package. The inner package is configured to provide an indication that the inner package has been opened after being closed. A prefilled syringe is disposed within the inner package. The syringe includes a barrel comprising a polymeric material and a dose of the injectable pharmaceutical composition.

### BACKGROUND

Injectable pharmaceutical compounds are often packaged in a vial and then converted into a syringe for administration to a patient. A pre-filled syringe of the same compounds provides convenience in preparation (the volume of these products used within the operating and emergency rooms, as well as the intensive care unit, are very high) and reduces waste. It is important that these pre-filled syringes maintain the integrity of the compounds stored therein.

Known syringes with glass barrels are prone to breakage and delamination during storage. Known syringes with plastic, e.g., polypropylene, barrels often do not adequately protect oxygen-sensitive pharmaceutical compounds from exposure to oxygen that occurs when ambient oxygen diffuses through the barrel. Pharmaceutical compounds stored in known plastic syringes are also subject to water loss, further degrading the stability of the compounds. Plastic syringes can be packaged in blister packs, although these are bulky and require a lot of space. Plastic syringes can also be packaged in a foil pouch; although, the foil pouch interferes with the reading of an RFID on the syringe.

Propofol (2,6-diisopropylphenol) is a well-known intravenous anesthetic that is particularly sensitive to oxygen exposure. In order to provide suitable stability, syringes prefilled with propofol must be particularly resistant to oxygen permeation.

### SUMMARY

The present disclosure provides examples of a package containing syringe prefilled with a pharmaceutical composition. In an embodiment, a prepackaged injectable pharmaceutical composition includes a sealed outer package made of an oxygen impermeable material and an inner package disposed within the sealed outer package. The inner package is configured to provide an indication that the inner package has been opened after being closed. A prefilled syringe is disposed within the inner package. The syringe includes a barrel comprising a polymeric material and a dose of the injectable pharmaceutical composition. The sealed outer package isolates the pharmaceutical composition from ambient oxygen outside the sealed outer package.

In any embodiment, the pharmaceutical composition is propofol.

In any embodiment, the barrel comprises at least one of a cyclic olefin polymer and of a cyclic olefin copolymer.

In any embodiment, the prepackaged injectable pharmaceutical composition further comprises an oxygen adsorber disposed within the outer package.

In any embodiment, the outer package is filled with an inert gas prior to sealing.

In any embodiment, the inert gas is nitrogen.

In any embodiment, the prepackaged injectable pharmaceutical composition further comprises at least one additional inner package disposed within the sealed container, each of the at least one additional inner package having a prefilled syringe disposed therein.

In any embodiment, the prepackaged injectable pharmaceutical composition further comprises an RFID tag coupled to the barrel and configured to transmit information about the prefilled syringe.

In any embodiment, the outer package is light impermeable.

In any embodiment, the outer package further includes a foil layer configured to isolate an interior portion of the outer package from ultraviolet radiation.

In any embodiment, the foil layer comprises aluminum.

In any embodiment, an interior portion of the inner package is in fluid communication with an interior portion of the outer package.

An embodiment of disclosed a method of packaging a dose of an injectable pharmaceutical composition, the method comprising the steps of filling a syringe with a dose of the pharmaceutical composition, wherein the syringe comprises at least one of a cyclic olefin copolymer and a cyclic olefin polymer. The method further comprises the steps of inserting the syringe into an inner package configured to provide an indication that the inner package has been opened after being closed and closing the inner package. The method also comprises the step of sealing the inner package inside an outer package, wherein the outer package comprises an oxygen impermeable material.

In any embodiment, the pharmaceutical composition is propofol.

In any embodiment, the method further comprises a step of inserting an oxygen adsorb within the outer package prior to sealing the outer package.

In any embodiment, the sealed outer package is light impermeable.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

The foregoing aspects and many of the attendant advantages of the disclosed subject matter will become more readily appreciated as the same become better understood by reference to the following detailed description, when taken in conjunction with the accompanying drawings, wherein:

FIG. 1 shows a cross-sectional side view of a syringe filled with a pharmaceutical composition according to a representative embodiment of the present disclosure; and

FIG. 2 shows an exploded view of an exemplary container for storing the syringe of FIG. 1;

FIG. 3 shows an exploded view of another exemplary container for storing the syringe of FIG. 1;

FIG. 4 shows a chart of stability of propofol repackaged according to aspects of the present disclosure; and

FIG. 5 shows a chart of stability of propofol repackaged according to aspects of the present disclosure.

### DETAILED DESCRIPTION

The detailed description set forth below in connection with the appended drawings, where like numerals reference like elements, are intended as a description of various embodiments of the present disclosure and are not intended to represent the only embodiments. Each embodiment described in this disclosure is provided merely as an example or illustration and should not be construed as preferred or advantageous over other embodiments. The illustrative examples provided herein are not intended to be exhaustive or to limit the disclosure to the precise forms disclosed. Similarly, any steps described herein may be interchangeable with other steps, or combinations of steps, in order to achieve the same or substantially similar result.

Embodiments of the disclosed subject matter is directed to a syringe suitable for filling with a pharmaceutical composition, such as propofol, prior to being packaged and shipped to an end user. The disclosed syringe and packaging provide improved resistance to oxygen exposure, light exposure, and water loss, thereby improving the stability of the compositions delivered therein.

In the case of propofol, the embodiments of the disclosed structure and packaging of the syringe maintain the stability of the propofol for extended periods but in any event, greater than or equal to about twelve months within the packaging. The syringe outside of the packaging has a shorter shelf life than within the packaging, e.g., eight months or less for some embodiments, and twelve months or less for some embodiments. Further, disclosed syringe and packaging can be terminally sterilized and can maintain sterility for a period of two years.

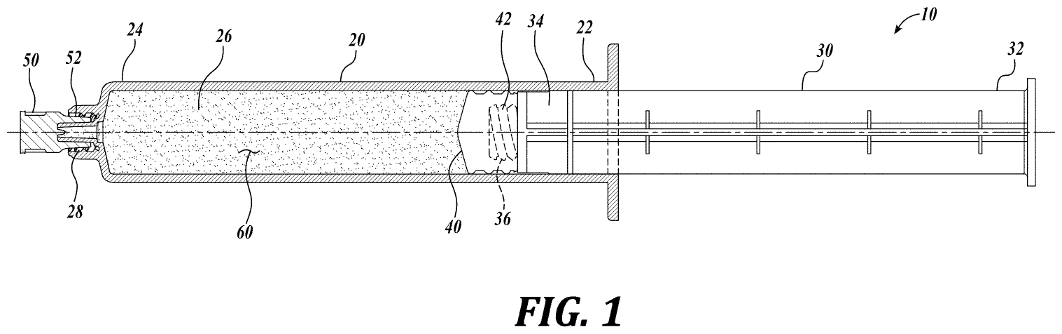

Referring to FIG. 1, a representative embodiment of a syringe 10 is shown. The syringe 10 includes a barrel 20 having an elongate cylindrical shape. A first end 22 of the barrel 20 includes an aperture sized and configured to receive a plunger 40. The plunger 40 is elastic and slidable relative to a longitudinal axis of the barrel while maintaining sealing engagement with an inner wall of the barrel 20. An elongate plunger rod 30 has a flanged first end 32 and a second end with a threaded portion 36 extending along an axial direction. The threaded portion 36 engages a threaded recess 42 formed in the plunger 40 to secure the rod 30 to the plunger 40.

A second end 24 of the barrel 20 includes an aperture extending through a threaded recess 28. A cap 50 includes a threaded extension 52 sized and configured such that the cap 50 is removably couplable to the threaded recess 28.

When the cap 50 is coupled to the barrel, the aperture in the second end 24 of the barrel 20 is sealed so that the barrel 20, the plunger 40, and the cap 50 cooperate to define a sealed cavity 26 that receives and stores a pharmaceutical composition 60. When the cap 50 is removed, pressing on the flanged end of the rod 30 discharges the pharmaceutical composition 60 from the aperture in the second end 24 of the barrel 20. The threaded recess 28 is sized and configured to have a needle coupled thereto, to engage a fitting of an intravenous line, and/or to engage any other suitable device or fitting suitable for use in the administration of a dose of the pharmaceutical composition 60.

The syringe barrel 20 is the primary container for drug integrity. In some embodiments, the barrel 20 is molded or otherwise formed from one or more polymeric materials, such as cyclic olefin copolymers (COC). COC materials are amorphous polymers that provide improved resistance to oxygen transmission as compared to polypropylene. COC also has suitable moisture barrier capabilities to prevent unacceptable water loss from the pharmaceutical composition 60. The nonreactive surfaces of COC materials do not degrade the pharmaceutical compositions 60, and the COC materials are compatible with known sterilization processes. In some embodiments, the barrel 20 is molded or otherwise formed from a cyclic olefin polymer (COP), which has physical properties similar to COC materials. In some embodiments, the barrel 20 is composed of polypropylene, polyethylene, or ethyl vinyl alcohol (EVOH). In some embodiments, the barrel 20 material is any material suitable for a single use, sterile, and non-pyrogenic prefillable polymer syringe.

The plunger 40 is molded or otherwise formed from an elastic, generally non-reactive rubber compound. In some embodiments, the plunger 40 is formed from a rubber formulation, wherein the rubber substrate is FM457/0, a bromobutyl rubber formulation with silicate filler. In some embodiments, the plunger is formed from a rubber formulation, wherein the rubber substrate is FM257, a bromobutyl rubber formulation with silicate filler. In some embodiments, the plunger is formed from a rubber formulation, wherein the rubber substrate is a chlorobutyl rubber formulation. In some embodiments, the plunger 40 is made of polypropylene entirely with no rubber compound. In some embodiments, the plunger is made of polyethylene entirely with no rubber compound.

The cap 50 is molded or otherwise formed from an elastic, generally non-reactive rubber compound. In some embodiments, the cap 50 comprises a rubber formulation, wherein the rubber substrate is FM257, a bromobutyl rubber formulation with silicate filler. In some embodiments, the cap 50 comprises a rubber formulation, wherein the rubber substrate is FM457/0, a bromobutyl rubber formulation with silicate filler. In some embodiments, the cap 50 comprises a rubber formulation, wherein the rubber substrate is a chlorobutyl rubber formulation. In some embodiments, the cap 50 comprises a polypropylene housing with or without a rubber compound.

The disclosed embodiments of a syringe 10 provide a barrier that prevents water loss from pharmaceutical compounds stored therein. The syringe 10 also provides limited oxygen permeability that protects the pharmaceutical compounds from unwanted exposure to oxygen. However, for products that are particularly sensitive to oxygen, such as propofol, it may be desirable to have additional protection against oxygen exposure.

Prior to packaging, the syringe 10 is prefilled with a pharmaceutical compound by any suitable process. Known processes for transferring pharmaceutical compounds from manufacturer vials to syringes maintain sterile and limit exposure of the pharmaceutical compounds to oxygen, water, and other substances that can negatively impact the efficacy of the pharmaceutical compounds.

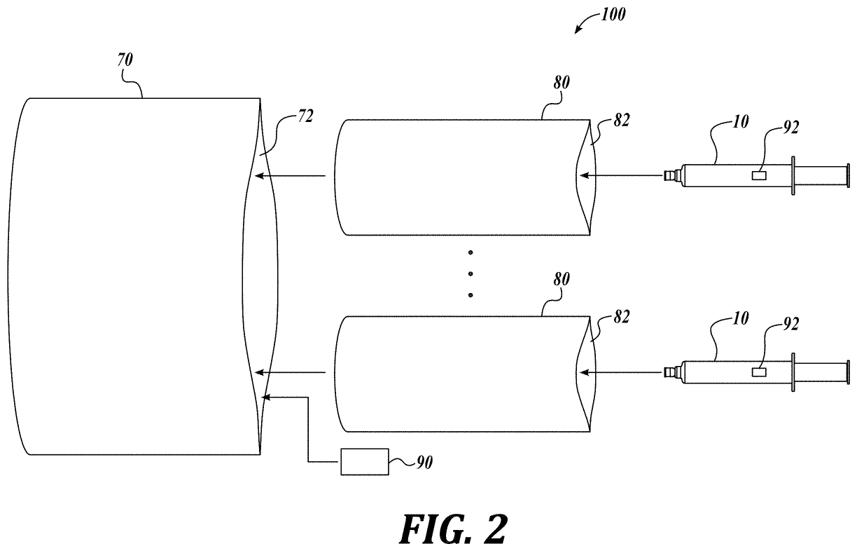

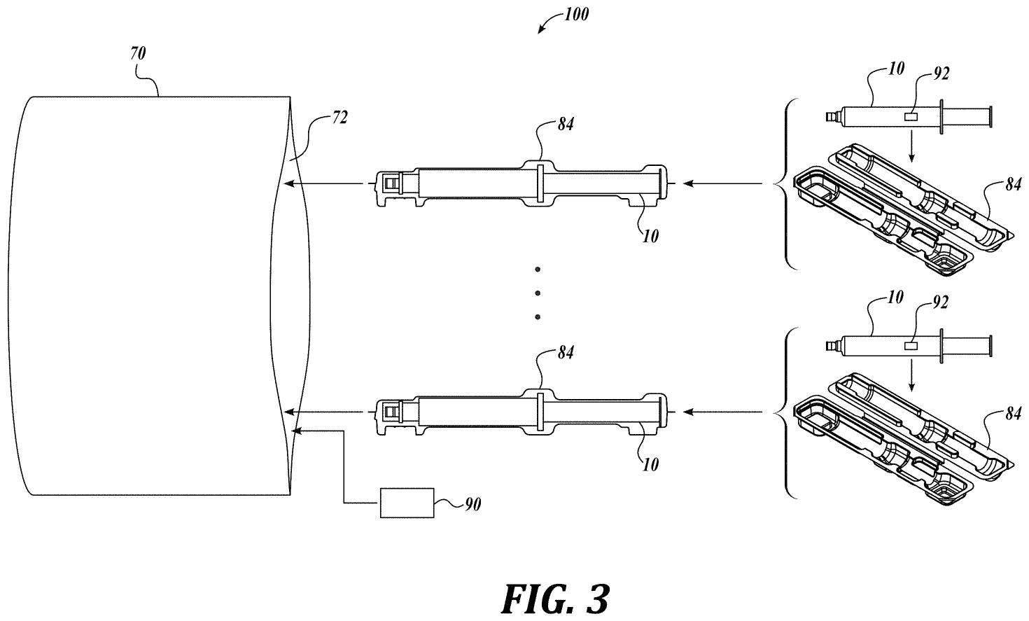

Referring now to FIGS. 2 and 3, various embodiments of a container 100 (packaging) is shown for storing multiple syringes 10 of FIG. 1 in order to provide additional stability of the pharmaceutical compound 60 stored in the syringe. More specifically, the described embodiments of the container 100 provide additional protection against oxygen exposure, light exposure, and tampering. As will be described in further detail, embodiments of the container 100 includes inner packaging, which provides evidence of tampering with the syringes 10, in conjunction with outer packaging, which provides an oxygen-free or low-oxygen environment for inner packaging, while also providing protection from light and, in particular, ultraviolet (UV) light.

In the embodiments shown in FIGS. 2 and 3, the container 100 includes a pouch 70 that acts as outer packaging of the container. The pouch 70 comprises a plastic film that is impermeable to oxygen or has a low oxygen transmission rate (OTR). As used herein, “low oxygen transmission rate” means an OTR of less than 15.5 cc/m²/24 hr as tested in accordance with ASTM-D3985. In some embodiments, the pouch 70 comprises a laminated material with a plastic film layer and a foil layer and has an OTR of less than 0.02 cc/m²/24 hr. Accordingly, the pouch 70 is less oxygen permeable, i.e., has a lower OTR, than those materials considered to be “semi-permeable” by one of ordinary skill in the art and identified by the U.S. Food and Drug Administration.

In some embodiments, the plastic film is a polyester film, such as BoPET (biaxially-oriented polyethylene terephthalate). In some embodiments, the pouch 70 is formed from or includes a metallic foil, an opaque material, or any other suitable material or combination of materials that provide a light-impermeable barrier. The pouch 70 is open at one end 72 to allow the syringes 10 to be placed therein. After the syringe 10 is placed in the pouch, the open end 72 is hermetically sealed so that the syringe is disposed within a generally oxygen and moisture impermeable container. In some embodiments, the pouch 70 is comprised of a plastic film with oxygen adsorption capability. In some embodiments, the pouch 70 is a flexible pouch. In some embodiments, the pouch 70 is a rigid or semi-rigid container. In some embodiment, the pouch 70 is any suitable shape or size. In this regard, the pouch may be sized and configured to receive multiple syringes 10 or any suitable number of syringes, including but not limited to any number of syringes from two to fifty or more. In some embodiments, the pouch 70 includes a foil layer to prevent or limit the transmission of UV light through the pouch 70.

Due to the volume of essential injectable products used in the clinical setting, distribution of products in bulky, individually packaged forms can limit storage capability at the point of care, leading to loss of efficiency provided by a syringe format. In some embodiments, the plunger 40 and plunger rod 30 are packaged uncoupled from each other, so that an end user attaches the plunger rod to the plunger prior to administering the pharmaceutical compound.

In some embodiments, the container 100 includes additional protection against oxygen permeation. In some embodiments, the pouch is at least partially filled with air, nitrogen, one or more inert gases, or any other suitable gas before being hermetically sealed. In some embodiments, an oxygen adsorber 90 is inserted into the pouch 70 with the syringes 10 to adsorb oxygen within the pouch. In some embodiments, the inclusion of an oxygen adsorber 90 reduces the oxygen level within the pouch to less than 1%.

In some embodiments, each of the syringes 10 is inserted into inner packaging, such as an inner sleeve 80, that itself is inserted into the pouch 70 prior to the pouch being sealed. As shown in FIG. 2, each syringe 10 may be inserted into an inner sleeve 80. In other embodiments, more than one syringe 10 may be inserted into each sleeve 80. In some embodiments, the inner sleeve 80 provides a barrier between the syringe 10 and ambient light, thereby protecting pharmaceutical compounds 60 that are light-sensitive. In some embodiments, the inner sleeve 80 is a pouch with an opening at one end 82. In some embodiments, the inner sleeve 80 is a blister pack with an opening. In some embodiments, the open end 82 of the inner sleeve 80 is sealed before being inserted into the pouch 70. In some embodiments, the inner sleeve 80 is formed from a metallic foil, an opaque material, or any other suitable material or combination of materials that provide a light-impermeable barrier. In some embodiments, the inner sleeve 80 is integrally formed with the pouch 70. In this regard, the pouch 70 may comprise a plurality of layers, wherein different layers are formed from different materials that provide different types of protection to the interior of the container 70.

Still referring to FIG. 2, an identification tag 92 is optionally attached to the syringe 10. The identification tag may be a radio-frequency identification (RFID) tag or a near field communication (NFC) tag that allows wireless, contactless communication for product identification and/or inventory tracking and control. In some embodiments, the identification tag 92 is attached to any suitable portion of the container 100. In some embodiments, the identification tag 92 is a bar code or other optical identifier. It will be appreciated that the type and placement of the identification tag may vary, and such variations should be considered within the scope of the present disclosure.

Referring now to FIG. 3, another embodiment is shown in which each of the syringes 10 is inserted into an open clamshell package 84, which is then closed. In the illustrated embodiment, clamshell package 84 has a unitary construction and is formed from a thermoplastic material by known thermoforming processes. In some embodiments, the clamshell package 84 is assembled from discrete components. In some embodiments, the clamshell package 84 and/or tray components are formed from any suitable material or combination of materials.

The inner packaging, such as the illustrated inner sleeve 80 and clamshell package 84, is configured such that once closed, opening the package provides a visual indication that the inner package has been opened, even if the inner package is subsequently closed or sealed again. In some embodiments, the opening the inner package requires tearing or cutting the inner package. In some embodiments opening the inner package ruptures a tamper-proof seal. In some embodiments, opening the inner package breaks a frangible portion of the packaging. In some embodiments, the inner packaging optionally provides additional resistance to UV transmission to further protect the syringes from exposure to UV light.

Disclosed embodiments of the syringe 10 used in combination with the container 100 provide improved product integrity and stability for pharmaceutical compounds. Embodiments of the syringe 10 can be sterilized, for example by terminal sterilization or aseptic manipulation, and can maintain sterility for a period of two years. The syringe 10 also advantageously provides a barrier that protects the pharmaceutical products against water loss. When sealed within the container 100, the syringe 10 is effectively isolated from ambient oxygen, and the stability of oxygen-sensitive compounds is increased.

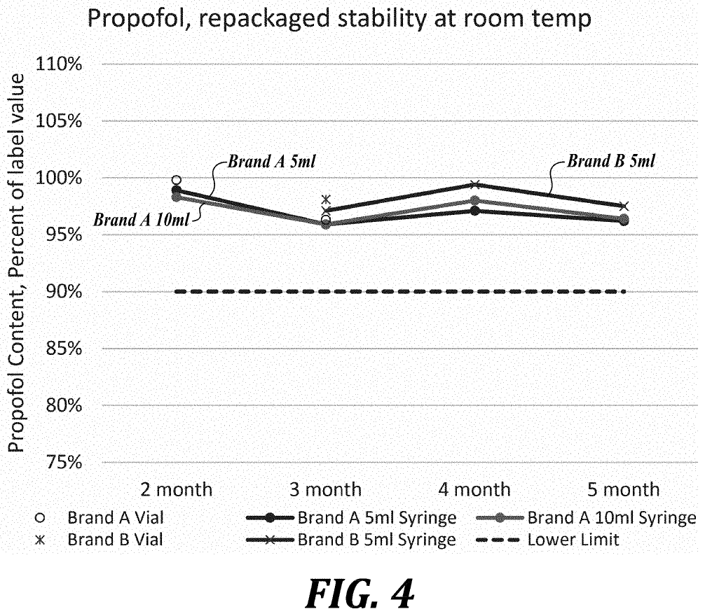

FIG. 4 shows the results of a stability study conducted for a product in a pre-filled syringe. A development stability study was performed with 5 ml and 10 ml syringes that had filled with different brands of propofol. As shown in FIG. 4, test results at five months shows the product remained with specification for both brands in both syringe sizes.

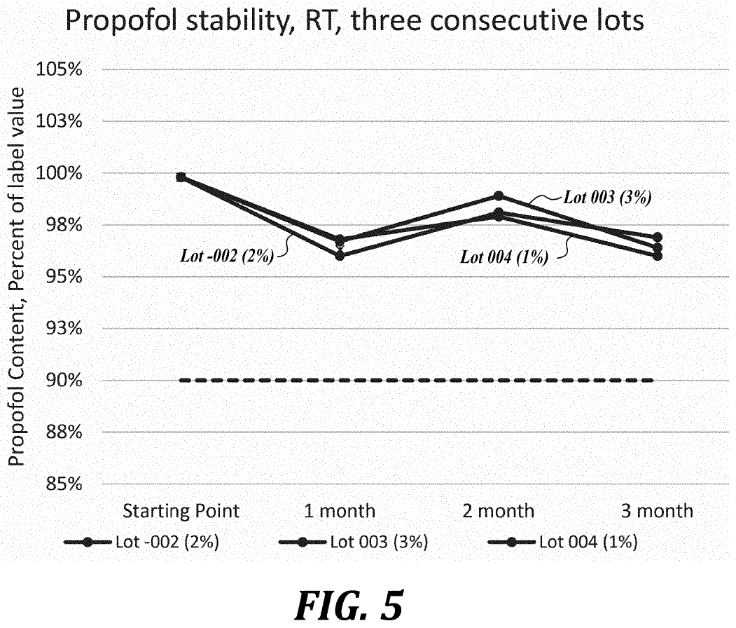

FIG. 5 shows the results of subsequent stability studies, wherein three subsequent lots of propofol were manufactured and packaged in 10 ml syringes and placed on stability. The three lots were manufactured using licensed drug product at the end of shelf life as a worst-case representation for future product. As shown in FIG. 5, repackaged propofol maintained stability for at least three months.

Applicant has further determined that propofol, when stored in the syringe 10 and sealed within the container 100 according to the present disclosure, remains stable for at least two to three years prior to the outer packaging being opened, and least eight months or longer after the outer package is opened. While aspects of the present disclosure have been described as being particularly effective for use with propofol, it will be appreciated that such use is exemplary only and should not be considered limiting. In this regard, the embodiments of the disclosed syringe 10, alone or in combination with a container 100, may be used with any suitable pharmaceutical compound, and such uses should be considered within the scope of the present disclosure.

Many oxygen-sensitive pharmaceuticals and propofol in particular are currently stored in glass vials or prefilled glass syringes. In this regard, known plastic syringe barrels do not provide suitable oxygen impermeability to maintain stability of the pharmaceuticals. By utilizing embodiments of a syringe and packaging described herein, less expensive, more durable plastic syringes can be pre-filled with oxygen-sensitive pharmaceuticals, while the outer packaging provides suitable isolation from ambient oxygen. At the same time, the inner packaging provides evidence of tampering and, optionally, additional UV protection.

In the foregoing description, specific details are set forth to provide a thorough understanding of exemplary embodiments of the present disclosure. It will be apparent to one skilled in the art, however, that the embodiments disclosed herein may be practiced without embodying all of the specific details. In some instances, well-known materials, configurations, and process steps may not have been described in detail in order not to unnecessarily obscure various aspects of the present disclosure. Further, it will be appreciated that embodiments of the present disclosure may employ any combination of features described herein.

The present application may reference quantities and numbers. Unless specifically stated, such quantities and numbers are not to be considered restrictive, but exemplary of the possible quantities or numbers associated with the present application. Also, in this regard, the present application may use the term “plurality” to reference a quantity or number. In this regard, the term “plurality” is meant to be any number that is more than one, for example, two, three, four, five, etc. The term “about,” “approximately,” etc., means plus or minus 10% of the stated value.

Throughout this specification, terms of art may be used. These terms are to take on their ordinary meaning in the art from which they come, unless specifically defined herein or the context of their use would suggest otherwise.

The principles, representative embodiments, and modes of operation of the present disclosure have been described in the foregoing description. However, aspects of the present disclosure, which are intended to be protected, are not to be construed as limited to the particular embodiments disclosed. Further, the embodiments described herein are to be regarded as illustrative rather than restrictive. It will be appreciated that variations and changes may be made by others, and equivalents employed, without departing from the spirit of the present disclosure. Accordingly, it is expressly intended that all such variations, changes, and equivalents fall within the spirit and scope of the present disclosure as claimed.

The embodiments of the invention in which an exclusive property or privilege is claimed are defined as follows:

1. A prepackaged injectable pharmaceutical composition, comprising:
a sealed outer package comprising a material with a low oxygen transmission rate;
an inner package disposed within the outer package, wherein the inner package is configured to provide an indication that the inner package has been opened after being closed; and
a prefilled syringe disposed within the inner package, the syringe comprising:
a barrel comprising a polymeric material; and
a dose of the injectable pharmaceutical composition.

2. The prepackaged injectable pharmaceutical composition of claim 1, wherein the pharmaceutical composition is propofol.

3. The prepackaged injectable pharmaceutical composition of claim 1, wherein the barrel comprises at least one of a cyclic olefin polymer, a cyclic olefin copolymer, and polypropylene.

4. The prepackaged injectable pharmaceutical composition of claim 1, wherein the sealed outer package has an oxygen transmission rate of less than 0.02 cc/m²/24 hr.

5. The prepackaged injectable pharmaceutical composition of claim 1, further comprising an oxygen adsorber disposed within the outer package.

6. The prepackaged injectable pharmaceutical composition of claim 1, wherein the outer package is filled with an inert gas prior to sealing.

7. The prepackaged injectable pharmaceutical composition of claim 6, wherein the inert gas is nitrogen.

8. The prepackaged injectable pharmaceutical composition of claim 1, further comprising at least one additional inner package disposed within the sealed container, each of the at least one additional inner packages having a prefilled syringe disposed therein.

9. The prepackaged injectable pharmaceutical composition of claim 1, further comprising an RFID tag coupled to the barrel and configured to transmit information about the prefilled syringe.

10. The prepackaged injectable pharmaceutical composition of claim 1, wherein the outer package further includes a foil layer.

11. The prepackaged injectable pharmaceutical composition of claim 10, wherein the foil layer comprises aluminum.

12. The prepackaged injectable pharmaceutical composition of claim 1, wherein an interior portion of the inner package is in fluid communication with an interior portion of the outer package.

13. A method of packaging a dose of an injectable pharmaceutical composition, the method comprising the steps of:
filling a syringe with a dose of the pharmaceutical composition, the syringe comprising at least one of a cyclic olefin copolymer, a cyclic olefin polymer, and polypropylene;
inserting the syringe into an inner package configured to provide an indication that the inner package has been opened after being closed;
closing the inner package; and
sealing the inner package inside an outer package, the outer package comprising a material with a low oxygen transmission rate.

14. The method of claim 13, wherein the pharmaceutical composition is propofol.

15. The method of claim 13, further comprising a step of inserting an oxygen adsorber within the outer package prior to sealing the outer package.
